(19) United States
(12) Patent Application Publication
(10) Pub. No.: US 2008/0021321 A1
(43) Pub. Date: Jan. 24, 2008

(54) Contrast agent destruction or therapy event indication in ultrasound medical imaging

(75) Inventors: Ismayil M. Guracar, Redwood City, CA (US); Anming He Cai, San Jose, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/439,483

(22) Filed: May 22, 2006

(51) Int. Cl. A61B 8/00; A61N 7/00

(52) U.S. Cl. 600/439; 600/458; 601/2

(57) ABSTRACT

Destructive or therapy events are indicated in medical ultrasound imaging. Audible, visual, vibratory or other feedback is provided to the user. The feedback indicates the use of the destructive or therapy events. A characteristic of the indicator may provide further information, such as the power level or other operating condition.

### BACKGROUND

The present embodiments relate to medical ultrasound imaging. In particular, contrast agent destruction events or therapy events are provided during ultrasound medical imaging.

Diagnostic ultrasound imaging may be used in conjunction with therapy. For example, a same ultrasound system generates acoustic energy for therapy and imaging. The acoustic energy for therapy heats tissue, moves tissue, destroys contrast agents, destroys contrast agents containing medication, causes cavitation or other causes therapeutic interaction. The imaging allows the user to guide the therapy.

In one form of therapy, acoustic energy which destroys contrast agents is periodically transmitted. For example, the destructive acoustic energy is transmitted at the R-wave of each heart cycle or based on other timing. At other portions of the heart cycle, ultrasound images are generated with lower power or less destructive acoustic energy. The response of the contrast agents to the lower power acoustic energy may assist in imaging or diagnosis. However, the periodic destruction of the contrast agents may prevent adequate reperfusion of the contrast agents into an imaged area. If the user fails to remember or recognize that the contrast agent destructive pulses continue to be periodically transmitted, the imaging benefits of the contrast agents may not be provided. Some imaging modes isolate returns from contrast agents. Without the contrast agents, little imaging information may be generated.

Various indications may be provided to a user. For example, an “on” light may be activated for ultrasound heat therapy machines. As another example, a lithotripsy system generates a load bang sound from the spark-gap transducer. Audio may indicate Doppler imaging on an imaging ultrasound system. A textual reference may indicate user activation of a destruction frame in response to depression of a button on an imaging ultrasound system.

### BRIEF SUMMARY

By way of introduction, the preferred embodiments described below include methods, systems, and computer readable media for indicating destructive or therapy events in medical ultrasound imaging. Audible, visual, tactile or other feedback is provided to the user. The feedback indicates the use of the destructive or therapy events. A characteristic of the indicator may provide further information, such as the power level or other operating condition.

In a first aspect, a computer readable storage medium has stored therein data representing instructions executable by a programmed processor for destructive or therapy events in medical ultrasound imaging. The storage medium includes instructions for imaging a patient with an ultrasound system, generating, in response to a trigger other than user activation, a therapy or contrast agent destruction transmission with the ultrasound system, and indicating a therapy or contrast agent destruction event separate from any imaging indication in information representing the scanned region.

In a second aspect, a method is provided for contrast agent destruction event indication in medical ultrasound imaging. Contrast agents are imaged at about a first power. Contrast agent destructive pulses are generated at about a second power greater than the first power. A speaker generates an audible indicator of the contrast agent destructive pulses.

In a third aspect, a method is provided for event indication in medical ultrasound imaging. A patient is imaged with ultrasound. An audible indicator associated with therapy or contrast agent destruction is generated. The audible indicator has a characteristic which is a function of power, timing or combinations thereof of the therapy or contrast agent destruction.

In a fourth aspect, a system is provided for destructive or therapy events in medical ultrasound imaging. A transmit beamformer is operable to generate therapy or contrast agent destruction events with a transducer probe. A rumbler associated with the transducer probe is operable to vibrate in correspondence with the therapy or contrast agent destruction events.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for destructive or therapy event indication in medical ultrasound imaging;

FIG. 2 is a block diagram of one embodiment of a sound generator; and

FIG. 3 is a flow chart of one embodiment of a method for indicating contrast agent destruction events or therapy events in medical ultrasound imaging.

### DETAILED DESCRIPTION OF THE DRAWINGS AND PRESENTLY PREFERRED EMBODIMENTS

Diagnostic ultrasound systems are capable of generating therapeutic responses in the body, such as when used in conjunction with contrast agents (e.g., microbubbles). Imaging and destruction or therapy sequences operate together. To keep sonographers aware that high intensity ultrasound is being transmitted into the patient, feedback is provided. The feedback is audible, visual or tactile.

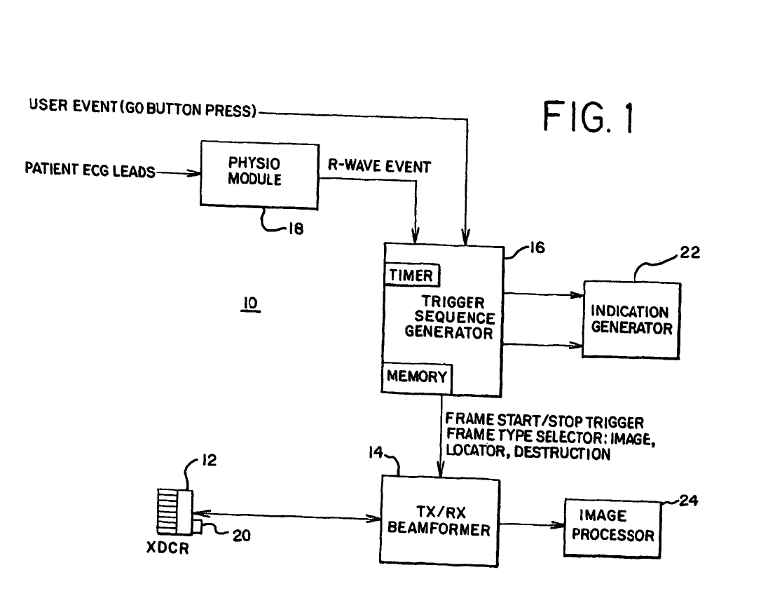

FIG. 1 shows one embodiment of a system 10 for destructive or therapy event indication in medical ultrasound imaging. The system 10 is part of a medical diagnostic ultrasound imaging system or a therapeutic ultrasound system with imaging capabilities. The system 10 includes a transducer probe 12, a beamformer 14, a trigger sequence generator 16, a physio module 18, a rumbler 20, an indication generator 22, and an image processor 24. Additional, different or fewer components may be provided. For example, the system 10 does not include the rumbler 20, or the system 10 includes the rumbler 20 as the indication generator 22.

The transducer probe 12 has a housing and an array of transducer elements. The housing is adapted or shaped for handheld use on the exterior of the patient. Alternatively, the housing is shaped as a catheter, intraoperative probe, intercavity probe, transesophageal probe or other now known or later developed transducer probe. The array is a linear, multidimensional, annular or other now known or later developed array of piezoelectric or microelectromechanical elements.

The transducer probe 12 operates for both therapeutic and diagnostic ultrasound imaging. For example, the same transducer probe 12 and array is used for generating acoustic wavefronts at different powers. Alternatively, separate arrays in a same housing or separate transducer probes 12 provide therapeutic ultrasound and the imaging ultrasound.

The transducer probe 12 generates acoustic energy in response to electrical signals from the beamformer 14. For imaging, acoustic echoes received by the transducer probe 12 are transduced into electrical signals, and the transducer probe 12 provides the electrical signals to the beamformer 14.

The beamformer 14 is a transmit beamformer, receive beamformer or both transmit and receive beamformer. As a transmit beamformer, the beamformer 14 includes waveform generators or pulsers, delays, phase rotators, timing generators, amplifiers, combinations thereof or other now known or later developed transmit beamformer components in a plurality of channels. For transmission, the beamformer 14 generates relatively delayed and apodized waveforms for each of a plurality of channels for a corresponding plurality of transducer elements. The transducer probe 12 forms an acoustic beam or beams in response to the waveforms. For therapy and/or contrast agent destruction, waveforms provide acoustic energy of about 1.0 MI or higher. The acoustic energy causes at least some contrast agents to burst or be absorbed. The frequency, power or other characteristic of the waveforms or acoustic beam may be set to provide the desired therapeutic effect. Therapy transmissions or pulses include pulses for destroying contrast agent, for heating tissue, for moving tissue, for causing cavitation or other therapy. Non-therapeutic transmissions may be used for destroying contrast agents. The pulses or transmissions are a therapy or contrast agent destruction event generated with the transducer probe 12.

For imaging contrast agents, acoustic energy with a lower mechanical index (MI) is generated. For example, acoustic energy of 0.7 MI or lower is used.

As a receive beamformer, the beamformer 14 includes channels with delays, phase rotators, amplifiers, or combinations thereof and includes a summer or summers for adding the signals from each channel together. For reception, the beamformer 14 generates samples representing different spatial locations. Samples for B-mode, flow mode, Doppler mode, spectral Doppler mode, harmonic imaging, contrast agent detection or other modes are generated. For example, different relative weightings and phase relationships of a transmit and/or receive sequence may be used to isolate the response of tissue, fluid, and/or contrast agents. U.S. Pat. Nos. 6,494,841, 6,602,195, 6,632,177, 6,638,228 and 6,682,482, the disclosures of which are incorporated herein by reference, show different amplitude and phase weightings to isolate information at particular frequency bands, such as isolating information at a cubic fundamental. Such information more likely may be from contrast agent than tissue or fluid.

The receive beamformed samples are provided to the image processor 24 for generating an image. The image processor 24 is a detector, filter, scan converter, three-dimensional processor, combinations thereof or other now known or later developed image generator. The detection is B-mode (intensity), flow-mode (velocity, energy, and/or variance), spectral Doppler and/or other detectors. The samples are detected, scan converted and provided to a display device.

The physio module 18 is an EKG device. Alternatively, the physio module 18 is a processor for receiving ultrasound data and determining a heart cycle from the ultrasound data. In other embodiments, the physio module 18 is a breathing detector. The physio module 18 generates signals or data representing a physiological cycle of a patient. In alternative embodiments, the physio module 18 is not provided, such as where triggering is performed with a timer.

The trigger sequence generator 16 is a processor, digital signal processor, controller, application specific integrated circuit, field programmable gate array, timer, analog circuit, digital circuit, combinations thereof, or other now known or later developed device for triggering generation of therapy or contrast agent destructive transmissions. The trigger sequence generator 16 receives the signals or data representing a physiological cycle, user activation input, combinations thereof or no input. For example, the trigger sequence generator 16 receives signals representing a heart cycle and identifies an event, such as the systole phase (R-wave), diastole phase, or other portion. As another example, the trigger sequence generator 16 receives user input to trigger. In another example, the trigger sequence generator 16 triggers based on time. The trigger sequence generator 16 receives internal or external events, such as the R-wave triggers or user control, and determines a sequence of ultrasound imaging and therapy or contrast agent destruction scans. The beamformer 14 operates based on the scan control from the trigger sequence generator 16.

The trigger sequence generator 16 also outputs signals to the indication generator 22. The output signals indicate the generation of the therapy or contrast agent destructive transmission. The trigger sequence generator 16 initiates generation of the indicator whenever the beamformer 14 is triggered to fire contrast agent destroying or therapy pulses. Alternatively, the beamformer 14 controls or outputs to the indication generator 22.

The indication generator 22 is a speaker, processor, pulser, waveform generator, combinations thereof or other now known or later developed device for generating an indication based on the transmission of therapy or contrast agent destructive pulses. The indication generator 22 generates an audible, visual, tactile or other indication. The indicator informs the user that the system is producing contrast agent destructive or therapy pulses. When using contrast agent imaging, the indicator reminds the user why contrast agents for imaging may be limited or not in the field of view.

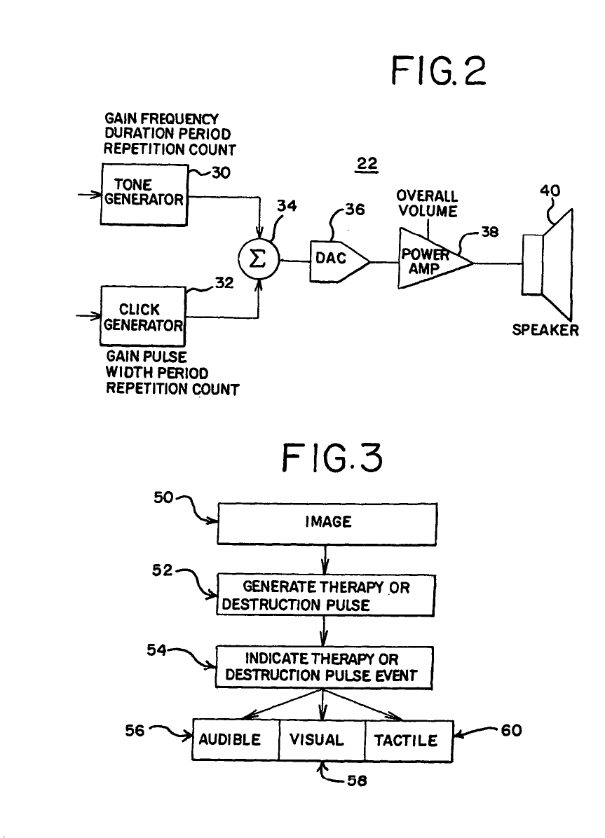

FIG. 2 shows one embodiment of the indication generator 22 for audible indication. A tone generator 30 generates a tone. A click generator 32 generates a click. Only one or more than two generators may be used. Separate generators 30, 32 allow for independent sound generation and/or different types of sounds for different conditions. The tone generator 30 generates tone signals. Any of various characteristics of the tone may be selected, such as the gain, frequency, duration, period, and/or repetition count. The click generator 32 generates click signals. Any of various characteristics of the clicks may be selected, such as the gain, pulse width, period and/or repetition count. Each characteristic is selectable or set (i.e., fixed). Noise burst, such as white noise, or voice-generated messages may be used.

An adder 34 sums the signals from each generator 30, 32. Where the generators 30, 32 generate digital representations of the desired sounds, a digital-to-analog converter 36 generates an analog signal. Alternatively, the generators 30, 32 generate analog signals. A power amplifier 38 controls the overall sound. A user or processor control sets the volume level. Independent amplifiers for controlling relative volume for different paths may be provided. The speaker 40 generates audible sound as an indicator. The speaker 40 is separate from the transducer probe 12 (FIG. 1) or the array (e.g., on the probe 12, but not part of the array). In one embodiment, the speaker 40 is a speaker used for other purposes in the system 10 (FIG. 1).

Referring to FIG. 1, the indication generator 22 may generate a visual indicator in other embodiments. For example, the indication generator 22 generates text, a symbol, color, blinking or other visual effect. A non-textual visual indicator (e.g., small blinking colored symbol, coloring text provided for other purposes, or blinking text provided for other purposes) is positioned in an annotation area of a screen. The visual effect is added to one or more displayed images. For example, a field of a display separate from an image representing the patient or scanned region includes the visual effect. In other embodiments, a separate display, such as a light, is used for the visual indication.

In another embodiment, the indication generator 22 is the rumbler 20 on the transducer probe 12 or elsewhere. The rumbler 20 is a vibrator, vibration motor, or other device for generating a vibration or tactile feedback. The rumbler 20 vibrates in correspondence with the therapy or contrast agent destruction events. The user holding the transducer probe 12 feels the vibration, indicating the continued transmission of the therapy or contrast agent destructive pulses.

The indicator has set characteristics, such as always generating a same tone. Alternatively, one or more characteristics of the indicator may be altered. Similarly, the indicator is generated in response to each trigger signal or other signal representing generation of the therapy or contrast agent destructive pulses. Alternatively, the indicator is generated independent of the rate of the trigger signal. The rate of trigger signal generation may be variable due to changes in the physiological cycle and/or variation in the sequence of therapy pulses. By providing for generation of the indication in response to but not at a same frequency of occurrence as the trigger signal, undesired indicators may be avoided. For example, the clicking rate and sound characteristics are independently controlled rather than being set based on the destruction or therapy frame rate. The sounds or other indications are set as desired, such as a soft clicking. A high-pitched or frequency tone may be undesired or desired as indicating an emergency in the medical environment.

The volume, frequency or other characteristic of the audible, visual, and/or vibratory indicators may be adjusted to indicate information for the user in addition to the ongoing transmissions. For example, the characteristic is determined as a function of a power level of the therapy or destruction pulses. If the power is set too low to achieve adequate destruction of contrast agents, a lower frequency tone or rate of clicking is used. Maximum or optimum power levels may be indicated with a more pleasant or quieter audible indication.

The indicator may be combined with other indicators. For example, an audible indicator of therapy or contrast agent destruction is combined with an audible indicator of a heartbeat event, such as the R-wave. The characteristics of the audible indicators are set differently to distinguish the events.

The trigger sequence generator 16 includes a memory, but the memory may be positioned, at least in part or entirely, elsewhere. The memory is a computer readable storage medium, such as a cache, buffer, RAM, removable media, hard drive or other computer readable storage media. Computer readable storage media include various types of volatile and nonvolatile storage media. Data representing instructions executable by a programmed processor for destructive or therapy event indication in medical ultrasound imaging is stored in the memory. The instructions are for implementing the processes, methods and/or techniques discussed herein. The functions, acts or tasks illustrated in the figures or described herein are executed in response to one or more sets of instructions stored in or on the computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

FIG. 3 shows a method for event indication in medical ultrasound imaging. For example, contrast agent destruction event indication is provided in medical ultrasound imaging. The method is performed by the system 10 of FIG. 1, the indication generator 22 of FIG. 1 or 2, or different systems or indication generators. Additional, different or fewer acts may be performed. For example, only one or two of the indication acts 56, 58, or 60 are performed.

In act 50, a patient is imaged with an ultrasound system. Ultrasound energy is used to scan a one, two or three-dimensional field of view of the patient. One or more images are generated from the scan. Where contrast agents are within the patient, the contrast agents may be imaged. For imaging contrast agents with minimized destruction, the power of the acoustic energy is low. For example, any transmitted beams have a mechanical index less than 1.0. Other higher or lower powers may be used. Imaging of the patient without contrast agents may be provided. Any now known or later developed modes of imaging may be used.

In act 52, a therapy or contrast agent destruction transmission is generated with the ultrasound system. The same ultrasound system and/or the transducer probe used for imaging are also used for generating the therapy or contrast agent destruction transmission. The transmission is intended to destroy contrast agents, or the destruction of contrast agents is a side effect. For example, therapy transmission for heating or moving tissue may have a power sufficient to destroy some or all of any contrast agents in a field of view. The pulses of the therapy or contrast agent destruction events may have a greater power than for imaging, such as greater than 1.0 MI. The therapy pulse may be generated in situations where contrast agents are not provided in other embodiments.

The therapy or contrast agent destruction transmission is for a same or different region than for imaging. For example, therapy is applied to a smaller region within the imaging field of view.

The therapy or contrast agent destruction pulse is generated in response to a trigger or other action. For example, user activation (e.g., depressing a button) causes immediate generation of a pulse. As another example, the pulse is generated in response to a trigger other than user activation. Such triggers include a heart cycle trigger, such as causing contrast agent destruction in response to every R-wave of a heart cycle. The user may input settings, such as configuring the ultrasound system for the desired therapy or contrast agent destruction sequence. The trigger event, such as based on a timer or physiological cycle, triggers the pulses based on the configuration, but the pulses are not triggered by the user.

In act 54, the therapy or contrast agent destruction event is indicated. The event is indicated directly, such as providing an indicator every time the event occurs. Alternatively, the event is indicated indirectly, such as providing an indicator that the therapy or contrast agent destruction is ongoing. The indicator is separate or different from any imaging indication in information representing the patient. For example, the disappearance of contrast agent echoes from an image of the patient may be seen in the image representing the patient. A separate visual indicator may be generated in another screen area, such as an annotation area.

In act 56, a speaker generates an audio indicator. The speaker is separate from the transducer probe, such as being a system speaker. The audio indicator may be any audible sound, such as a click, beep, chirp, noise burst or combination thereof.

In act 58, a visual indicator is generated. The visual indicator is on the display of the system or a separate display. Text or non-textual indication may be used. For non-textual visual indication, blinking, coloring or combinations thereof of textual, graphical or other symbols may be used.

In act 60, a tactile indicator is generated. For example, a transducer probe vibrates. Other devices may be vibrated. Other tactile indications may be used.

The indication has predetermined or has set characteristics. Alternatively, one or more characteristics are controlled as a function of a condition of the therapy or destructive pulses. Conditions include power (e.g., power level), timing (e.g., undesired timing), rate, quantity, strength or combinations thereof. The rate of transmission of the therapy or destruction pulses may vary based on configured timing or sensed physiological information, so the indicator also varies. Therapy may use a particular quantity of pulses, so the indicator varies as a function of the quantity provided so far in a given sequence. The strength of the therapy or destruction pulses, such as provided by the power level, may vary depending on the therapy or desired amount of contrast agent destruction. The timing of the therapy or destruction pulse may be, at least in part, independent of a physiological cycle. Pulses at certain points in the cycle may be undesired, so the indicator changes for such pulses.

The controlled characteristics are dependent on the type of indicator. For example, the tone frequency, frequency of the indicator occurrence, message and/or volume of an audible indicator vary as a function of the power or other condition. As another example, the brightness, frequency of blinking, shape, size, symbol, and/or text of a visual indicator vary as a function of power or other condition. In another example, the vibration frequency, and/or vibration power vary as a function of power or other condition.

A range of characteristics may reflect a range of the power or other conditions for the therapy or destructive pulses. Alternatively, distinguishing steps may be provided, such as altering the tone frequency, volume or other characteristic to reflect adjustment past a threshold. For example, setting therapy or destruction pulses to an insufficient power may be reflected by audible indication different from the audible indication for pulses with sufficiently high power.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. In a computer readable storage medium having stored therein data representing instructions executable by a programmed processor for destructive or therapy event indication in medical ultrasound imaging, the storage medium comprising instructions for:
imaging a patient with an ultrasound system;
generating, in response to a trigger other than user activation, a therapy or contrast agent destruction transmission with the ultrasound system; and
indicating a therapy or contrast agent destruction event separate from any imaging indication in information representing a scanned region of the patient.

2. The instructions of claim 1 wherein indicating comprises generating a non-textual visual indicator.

3. The instructions of claim 2 wherein generating the non-textual visual indicator comprises blinking, coloring or combinations thereof the non-textual visual indicator.

4. The instructions of claim 1 wherein indicating comprises generating an audio indicator with a speaker separate from a transducer of the ultrasound system.

5. The instructions of claim 4 wherein generating comprises generating a click, beep, chirp, noise burst or combination thereof.

6. The instructions of claim 1 wherein indicating further comprises indicating a power level, timing, rate, undesired timing, quantity, or combinations thereof of the transmission with the audio indicator.

7. The instructions of claim 6 wherein indicating comprises indicating with a frequency, a brightness, a volume or combinations thereof of the indication.

8. The instructions of claim 1 wherein indicating comprises indicating with a vibration.

9. The instructions of claim 8 wherein indicating with the vibration comprises vibrating a transducer probe.

10. The instructions of claim 1 wherein imaging and generating comprises imaging and generating with a same transducer, the generating comprising generating a contrast agent destruction transmission in response to a heart cycle trigger.

11. A method for contrast agent destruction event indication in medical ultrasound imaging, the method comprising:
imaging contrast agents at about a first power;
generating contrast agent destructive pulses at about a second power greater than the first power; and
generating an audible indicator of the contrast agent destructive pulses with a speaker.

12. The method of claim 11 wherein generating the audible indicator comprises generating a click, beep, chirp, noise burst or combinations thereof.

13. The method of claim 11 further comprising controlling a characteristic of the audible indicator as a function of power, timing, rate, quantity, strength or combinations thereof of the destructive pulses.

14. The method of claim 13 wherein controlling the characteristic comprises controlling a frequency, volume or combination thereof.

15. The method of claim 11 wherein generating the contrast agent destructive pulses comprises generating in response to a heart cycle trigger.

16. A method for event indication in medical ultrasound imaging, the method comprising:
imaging a patient with ultrasound; and
generating an audible indicator associated with therapy or contrast agent destruction, the audible indicator having a characteristic which is a function of power, timing or combinations thereof of the therapy or contrast agent destruction.

17. The method of claim 16 further comprising generating a pulse for the therapy or contrast agent destruction, wherein generating the audible indicator comprises generating the audible indication in response to generating the pulse.

18. The method of claim 16 wherein generating the audible indicator comprises generating the audible indicator with the characteristic comprising a tone frequency, a volume or combinations thereof.

19. The method of claim 16 wherein generating the audible indicator comprises generating the audible indicator having the characteristic which is a function of the power.

20. The method of claim 19 wherein the function of the power indicates an insufficient power.

21. The method of claim 16 wherein generating the audible indicator comprises generating the audible indicator having the characteristic with is a function of frequency.

22. The method of claim 16 wherein generating the audible indicator comprises generating the audible indicator having the characteristic which is a function of timing with respect to an event of a heart cycle.

23. A system for destructive or therapy event indication in medical ultrasound imaging, the system comprising:
a transducer probe;
a transmit beamformer operable to generate therapy or contrast agent destruction events with the transducer probe; and
a rumbler associated with the transducer probe, the rumbler operable to provide tactile feedback in correspondence with the therapy or contrast agent destruction events.
